(19) United States
(12) Patent Application Publication
(10) Pub. No.: US 2005/0171451 A1
(43) Pub. Date: Aug. 4, 2005

(54) System and method for managing growth and development of a user

(76) Inventors: Hyung-sok Yeo, Yongin-si (KR); Jin-sang Whang, Suwon-si (KR); Kyung-ho Kim, Yongin-si (KR); Jeong-hwan Lee, Suwon-si (KR)

Correspondence Address: LEE, STERBA & MORSE, P.C., SUITE 2000, 1101 WILSON BOULEVARD, ARLINGTON, VA, 22209, US

(21) Appl. No.: 11/049,946

(22) Filed: Feb. 4, 2005

(30) Foreign Application Priority Data
Feb. 4, 2004 (KR) 10-2004-0007232

(51) Int. Cl. A61B 5/05; A61B 5/04; A61B 5/00

(52) U.S. Cl. 600/547; 600/300; 600/509

(57) ABSTRACT

In a system and method for managing growth and development of a user, e.g., a child, the system includes a biological information measuring module for acquiring at least two biological signals to be used for analyzing growth and development of the user, and for identifying the user by analyzing at least one biological signal of the at least two acquired biological signals and a biological information processing module for evaluating a development state and a growth level of the user according to the biological signals, for storing and managing personal data and the results of the evaluation of the user.

### BACKGROUND OF THE INVENTION

1. Field of the Invention

The present invention relates to an apparatus and a method for measuring biological information. More particularly, the present invention relates to an apparatus and a method for measuring and managing biological information associated with growth and development of a user, e.g., a child.

2. Description of the Related Art

In general, biological information reflecting degrees of growth of people, e.g., children, include, e.g., height, weight, and the like, and biological information reflecting development states of people, e.g., children, include factors such as data on fatness, etc., which is obtained by measuring body fat of a person, e.g., a child. The data such as height, weight, and the like, which are used as barometers for evaluating a growth degree, are the most basic biological information reflecting the growth degrees of a human body. The body fat data, which is used as a barometer for evaluating a development state, is widely used as a parameter reflecting nutritive conditions of a human body. For example, the body fat data is also important as a parameter for managing personal appearance, i.e., being thin or obese, and can be also used for diagnosing the development of children and the nutritive conditions of people, e.g., children, the elderly or the infirm.

Generally, biological information can be obtained by measuring growth data and development data with individual measuring modules. However, conventional biological information measuring apparatuses are only to supply the biological information, such as weight, height, body fat, etc., individually, and not comprehensively. More specifically, conventional biological information measuring apparatuses do not supply the health data for managing growth and development states of children for periods of time (e.g., daily, weekly, monthly, etc.) and do not provide solutions, e.g., customized exercise data, diet, rest, etc., suitable for the development states of the individual.

Further, since most biological information measuring apparatuses operate manually, there are disadvantages in that a certain degree of error always exists. Furthermore, since conventional measuring apparatuses occupy large spaces, there is a disadvantage in that it is difficult to move and store the measuring apparatuses.

### SUMMARY OF THE INVENTION

The present invention is therefore directed to an apparatus and a method for measuring and managing biological information associated with growth and development of an individual user, e.g., a child, which substantially overcome one or more of the problems due to the limitations and disadvantages of the related art.

It is a feature of an embodiment of the present invention to provide a system and a method for managing growth and development of a user, e.g., a child, the system and the method comprehensively supplying data on a growth and development state of the child and solutions such as exercise, rest, diet, etc., suitable for a current condition of the child by collecting and analyzing biological information, e.g., height, weight, body fat, etc., of the child and showing time-sequential transition results in the same kind of biological information of the child, the data having been stored for a predetermined period.

It is another feature of an embodiment of the present invention to provide a system and a method for managing growth and development of a user, e.g., a child, which may be easily used at any location since a space for a biological signal measuring device is minimized to facilitate portability and storage of the biological signal measuring device.

It is still another feature of an embodiment of the present invention to provide a computer readable recording medium on which a program for executing the method with a computer is recorded.

At least one of the above features and other advantages may be provided by a system for managing growth and development of a user, the system including a biological information measuring module for acquiring at least two biological signals to be used for analyzing growth and development of the user, and for identifying the user by analyzing at least one biological signal of the at least two acquired biological signals, and a biological information processing module for evaluating a development state and a growth level of the user according to the biological signals, for storing and managing personal data and the results of the evaluation of the user.

The system may further include a data center for receiving biological information, the personal data, and the result of the evaluation of the development state and the growth level of the user from the biological information processing module, for analyzing information required for a health condition and health care of the user, and for supplying the health-relevant information of the user at predetermined periods.

The biological information measuring module may include a height measuring module for measuring a height of the user using ultrasonic waves, and an integrated identification-weight-body fat module for measuring an electrocardiogram, a weight, and a bioelectric impedance of the user, for identifying the user by analyzing the measured electrocardiogram, and for analyzing a body fat value from the bioelectric impedance.

The height measuring module may include a height data acquiring unit for applying the ultrasonic waves to a head portion of the user and for sensing ultrasonic waves reflected from the head portion of the user, an analog-to-digital converter for converting the sensed ultrasonic waves into digital data, a control unit for calculating the height of the user based on the digitized ultrasonic wave data, and a data transmitting unit for transmitting the calculated height data to the biological information processing module.

The identification-weight-body fat measuring module may include an identification module for identifying the user by comparing the measured electrocardiogram with previously-registered electrocardiogram templates, and a weight-body fat measuring module for measuring the weight and the bioelectric impedance of the user and for analyzing the body fat value from the measured bioelectric impedance.

The weight-body fat measuring module may include hand electrodes for contacting hands of the user and for acquiring the electrocardiogram and bioelectric impedance signal, foot electrodes for contacting feet of the user and for acquiring the electrocardiogram and bioelectric impedance signal, a load cell for contacting the feet of the user and acquiring a weight signal, a weight measuring unit for amplifying the weight signal acquired by the load cell, a body fat measuring unit for measuring the bioelectric impedance signal by flowing a predetermined amount of current through the hand electrodes and the foot electrodes and measuring voltages across the electrodes, and for amplifying the measured bioelectric impedance signal, an analog-to-digital converter for converting the amplified weight signal and the amplified bioelectric impedance signal into digital weight data and digital bioelectric impedance data, respectively, a control unit for calculating the weight of the user by averaging the digital weight data, and for calculating the body fat by analyzing the digital bioelectric impedance data, and a data transmitting/receiving unit for transmitting the weight and the body fat data calculated by the control unit to the biological information processing module and for receiving the personal data of the user from the biological information processing module.

The identification module may include an electrocardiogram measuring unit for amplifying the electrocardiogram acquired through the hand electrodes and the foot electrodes, wherein the electrocardiogram amplified by the electrocardiogram measuring unit are compared with the electrocardiogram templates in the control unit in order to identify the user, and the personal data is received by the data transmitting/receiving unit.

The biological information processing module may include a data receiving unit for receiving wirelessly the biological information transmitted from the biological information measuring module, a data storage unit for storing the received biological information for the user, a data operation unit for analyzing the biological information, for evaluating the development state and the growth level, and for analyzing tendencies of variations in growth and development of the user by period, a data display unit for outputting the analyzed result to the user, a digital input and output controller for controlling the data input and output of the data receiving unit, the data storing unit, the data operation unit, and the data display unit, and a power supply unit for supplying electric power to the biological information processing module.

The biological information processing module may further include a data transmitting unit for transmitting the biological information, the personal data, and the evaluated result of the development state and the growth level of the user to the data center, and a data receiving unit for receiving the health-relevant data supplied from the data center and for providing the health-relevant data to the user.

The biological information processing module may be provided in one selected from the group including a stand-alone type device, a personal computer, a mobile phone, a wrist watch, and a personal digital assistant.

The data center may include a health care database for storing the health care data for the user, and the health care data may be supplied to the biological information processing module by one selected from the group including a voice message, a short message service, and an electronic mail.

At least one of the above features and other advantages may be provided by a method of managing growth and development of a user, the method including determining whether the user is positioned at a correct position for measurement of biological information, identifying the user based on at least one biological signal acquired from an integrated identification-weight-body fat measuring module, retrieving previously registered data of the identified user and measuring biological information of the user, transmitting the measured biological information to a biological information processing module, and evaluating a development state and a growth level of the user in response to the biological information and storing the result of the evaluation of the user.

The method may further include transmitting the biological information, personal data, and the result of the evaluation of the development state and growth level of the user to a data center, and receiving and analyzing the biological information, the personal data, and the result of the evaluation of the development state and growth level of the user, and supplying the analyzed health-relevant information at a series of predetermined periods to the biological information processing module.

Evaluating the development state and a growth level of the user may include calculating a body mass index based on the biological information and analyzing a weight and a fatness based on a body mass index table, analyzing the weight and the fatness of the user based on bioelectric impedance measured from the user when the fatness is above a predetermined value, evaluating the growth level of the user based on average heights and average weights for each sex and age, determining a positive growth entry term and a positive growth term in consideration of an increment rate in height of the user, and analyzing variation of the biological information by period by analyzing a tendency of the analyzed results for a predetermined period.

After an age of the user is over six years old, the positive growth entry term may be represented by an inflection point where a slope of a height increment rate curve for a period of the latest six months is converted into a positive (+) value from a negative (−) value or zero (0).

The positive growth term may be a term when the slope of the curve maintains the positive slope for six months or longer or when the slope of the curve becomes steeper with respect to the positive growth entry term.

At least one of the above features and other advantages may be provided by a computer readable recording medium on which a program for executing any of the above-described methods is recorded.

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 illustrates a diagram of a system for managing growth and development of a user, e.g., a child, according to an exemplary embodiment of the present invention;

FIG. 2 is a block diagram schematically illustrating a configuration of the system shown in FIG. 1;

FIG. 3 is a block diagram illustrating in detail a height measuring module shown in FIGS. 1 and 2;

FIG. 4 illustrates a diagram of an identification-weight-body fat measuring module shown in FIGS. 1 and 2;

FIG. 5 is a block diagram illustrating in detail a configuration of the identification-weight-body fat measuring module shown in FIGS. 1 and 2;

FIG. 6 is a block diagram illustrating a configuration of biological information processing module according to an embodiment of the present invention;

FIG. 7 is a block diagram illustrating an overall configuration of a system for managing growth and development of a child to which the biological information processing module shown in FIG. 6 is applied;

FIG. 8 is a block diagram illustrating a configuration of a biological information processing module according to another embodiment of the present invention;

FIG. 9 is a block diagram illustrating a configuration of a fatness and nutritive condition managing system and health information managing system to which the biological information processing module shown in FIG. 8 is applied;

FIG. 10 illustrates a data flow between the biological information processing module and a data center shown in FIG. 9;

FIG. 11 is a flowchart illustrating a method of measuring and analyzing growth and development of a user, e.g., a child, according to an exemplary embodiment of the present invention;

FIG. 12 is a flowchart illustrating in detail an operation of determining a correct position of a user shown in FIG. 11;

FIG. 13 is a flowchart illustrating in detail an operation of identifying a user shown in FIG. 11;

FIG. 14 is a flowchart illustrating in detail an operation of measuring biological information shown in FIG. 11;

FIG. 15 is a flowchart illustrating in detail an operation of processing and analyzing data shown in FIG. 11;

FIG. 16 is a graph illustrating an operation of determining a positive growth term shown in FIG. 15; and

FIG. 17 illustrates screens for analyzing growth and development of a user, e.g., a child, according to an exemplary embodiment of the present invention.

### DETAILED DESCRIPTION OF THE INVENTION

Korean Patent Application No.10-2004-0007232, filed on Feb. 4, 2004, in the Korean Intellectual Property Office, and entitled: “System and Method for Managing Growth and Development of a Child,” is incorporated by reference herein in its entirety.

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. Like reference numerals and characters indicate like elements throughout.

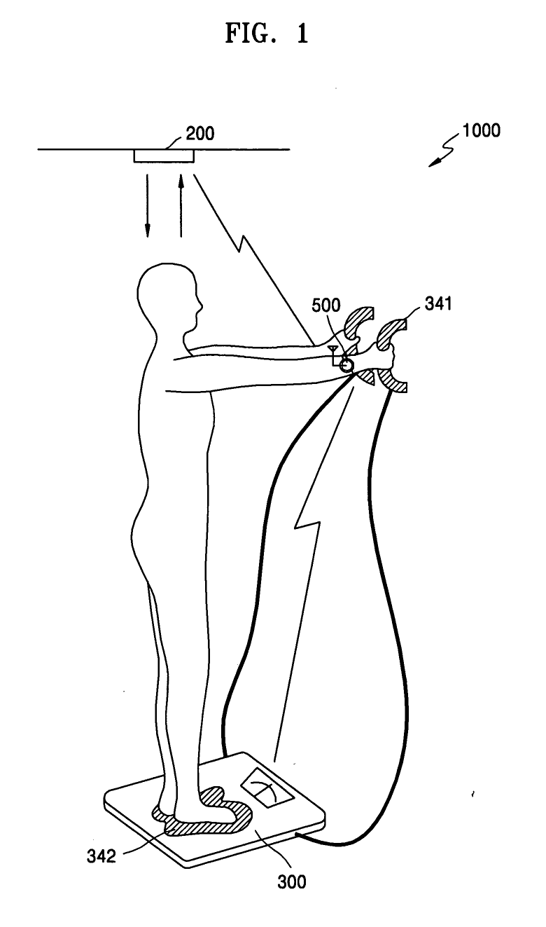

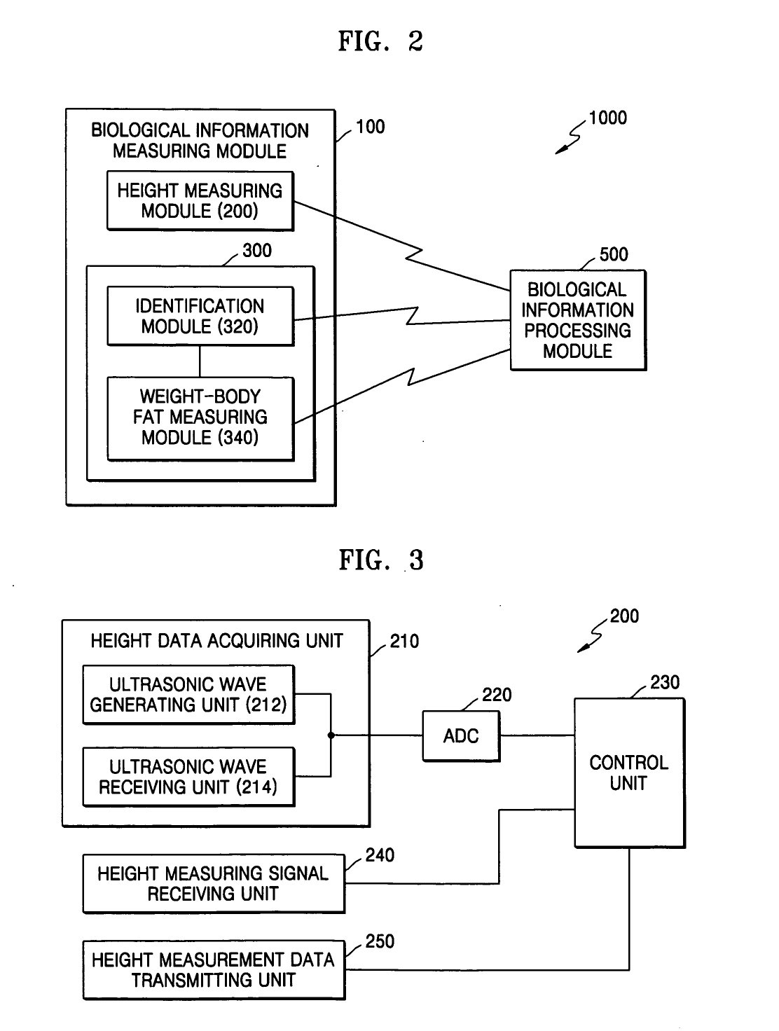

FIG. 1 illustrates a diagram of a system 1000 for managing growth and development of a user, e.g., a child, according to an exemplary embodiment of the present invention. FIG. 2 is a block diagram schematically illustrating a configuration of the system 1000 shown in FIG. 1.

Referring to FIGS. 1 and 2, the system 1000 for managing growth and development of a user, e.g., a child, generally includes a biological information measuring module 100 and a biological information processing module 500. The biological information measuring module 100 may include a height measuring module 200 and an identification-weight-body fat measuring module 300. The identification-weight-body fat measuring module 300 may include an identification module 320 and a weight-body fat measuring module 340. Since each of these modules may have a radio communication port, they are able to communicate with each other wirelessly when physically separated from one another.

The height measuring module 200 included in the biological information measuring module 100 measures a height of a user using ultrasonic waves, and transmits, e.g., wirelessly, the measured results by user to the biological information processing module 500. The identification-weight-body fat measuring module 300 analyzes electrocardiogram (ECG) signals acquired from hand electrodes 341 in contact with palms of the user and foot electrodes 342 in contact with soles of both feet of the user, while or before measuring the height with the height measuring module 200. In addition, the identification-weight-body fat measuring module 300 identifies the user. Then, weight and body fat of the identified user are comprehensively measured, and the measured results for each user are transmitted, e.g., wirelessly, to the biological information processing module 500. In this case, because the identification is automatically recognized through each of the analyzed ECG signals and information on the recognized user is transmitted to the biological information processing module 500, it is not necessary for a user to input individual data for registering whenever biological information is measured. Consequently, the biological information processing module 500 can systematically manage growth and development data for each user, including health-relevant information on the corresponding user.

The biological information processing module 500 receives the biological information transmitted from the biological information measuring module 100, e.g., via the radio communication port, analyzes the biological information, i.e., height, weight, body fat data, evaluates a development state and a growth level of the user, and analyzes growth and development variations for a series of predetermined periods. Then, the received biological information and the analyzed results are stored and managed in a predetermined storage area. The biological information processing module 500 may be a stand-alone type device, or may be embodied in a personal computer (PC), a mobile phone, a wrist watch, a personal digital assistant (PDA), or other similar device.

The system 1000 for managing growth and development of a user, e.g., a child, is portable and detachable and suitable for use at any location, e.g., at home or while traveling. Particularly, since the system 1000 may perform a function of measuring and managing data of children, it is constructed such that a basic measuring procedure is easy and simple and various biological information can be processed quickly and accurately through a batch procedure. Further, the system 1000 manages health-relevant data, e.g., fatness and nutritive condition, etc., for each user and each period, as well as managing growth and development of a child. Accordingly, the system can comprehensively supply additional information, e.g., health information, exercise information, diet, etc. for each period to each individual user, as will be further described in connection with FIG. 9. As noted above, the system is equally applicable to adults.

FIG. 3 is a detailed block diagram of the height measuring module shown in FIGS. 1 and 2. The height measuring module 200, which uses an ultrasonic wave sensor, is portable and can be easily set-up. For example, the height measuring module 200 may be placed virtually anywhere on a ceiling to measure a user's height.

Referring to FIG. 3, the height measuring module 200 may include a height data acquiring unit 210, an analog-to-digital converter (ADC) 220, a control unit 230, a height measuring signal receiving unit 240, and a height measurement data transmitting unit 250.

The height data acquiring unit 210 irradiates a user's head with ultrasonic waves using the Doppler effect and then senses the reflected ultrasonic waves. The height data acquiring unit 210 may include an ultrasonic wave generating unit 212 for irradiating the user with ultrasonic waves and an ultrasonic wave receiving unit 214 for receiving the ultrasonic waves reflected from the user.

The received analog-type ultrasonic waves acquired from the height data acquiring unit 210 are converted into a digital ultrasonic wave data by the ADC 220. The digital ultrasonic wave data is input to the control unit 230. The control unit 230 may include one of a microprocessor, a central processing unit (CPU), a digital signal processor (DSP), and another corresponding processor. The control unit 230 responds to height measuring signals received from the height measuring signal receiving unit 240 and the ultrasonic wave data, and calculates a user's height. After a user is positioned in a predetermined measuring position, the calculated user height is obtained by subtracting a distance from the ceiling to a top of the user's head from a distance from the ceiling to an upper surface of the identification-weight-body fat measuring module 300, which is positioned vertically below the height data acquiring unit 210. After the user's height is calculated by the control unit 230, it is transmitted, e.g., wirelessly, to the biological information processing module 500 by the height measurement data transmitting unit 250.

As described above, since the height measuring module 200 measures a user's height without contacting the user through the use of ultrasonic waves, errors and disadvantages can be reduced, as compared to a manual measurement. Also, a space occupied by the height measuring device may be reduced.

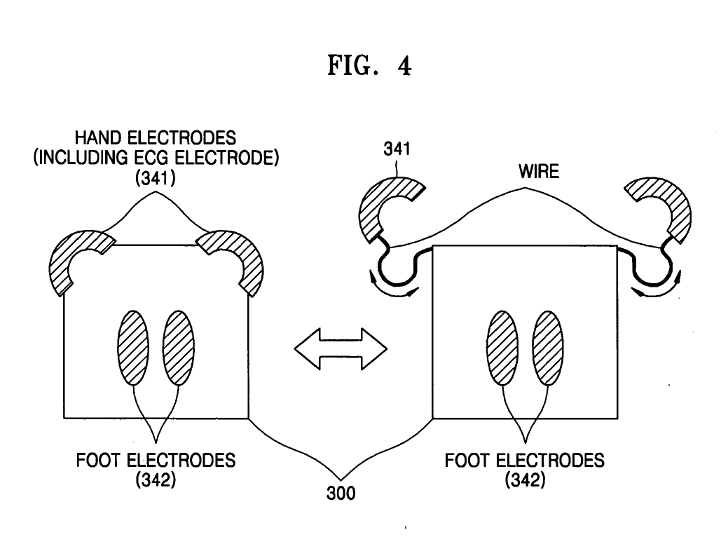

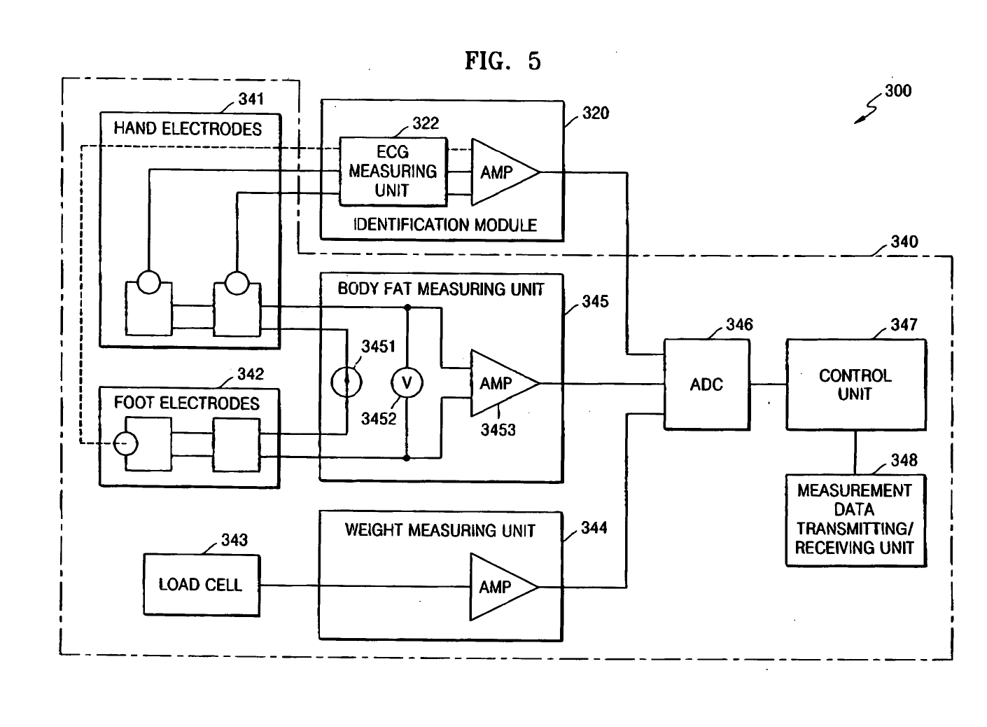

FIG. 4 illustrates a diagram of the identification-weight-body fat measuring module shown in FIGS. 1 and 2. FIG. 5 is a detailed block diagram illustrating a configuration of the identification-weight-body fat measuring module shown in FIGS. 1 and 2.

Referring to FIGS. 4 and 5, the identification-weight-body fat measuring module 300 is able to measure a user's weight, body fat, and ECG signals all at once. A load cell 343 for measuring weight is included in a main body of the identification-weight-body fat measuring module 300. Further, two foot electrodes 342 for measuring body fat are at a top portion of the main body of the identification-weight-body fat measuring module 300, and two hand electrodes 341 for measuring body fat are removably attached to two corners of the main body of the identification-weight-body fat measuring module 300.

The foot electrodes 342 are fixed to the top portion of the main body of the identification-weight-body fat measuring module 300, but the hand electrodes 341 are wired to the main body of the identification-weight-body fat measuring module 300 to provide flexibility of movement during a measurement. More specifically, when a user pulls the hand electrodes 341, a wire extends from the main body to each of the hand electrodes 341 so that the user may raise the hand electrodes 341 to the user's side. Further, when the user releases the hand electrodes 341, the wires connecting the hand electrodes 341 are retracted into the main body of the identification-weight-body fat measuring module 300 in order for the hand electrodes 341 to return to the corner positions of the main body.

The hand electrodes 341 and the foot electrodes 342 are commonly used for measuring body fat and ECG. When a user steps on the foot electrodes 342 and grasps the hand electrodes 341, the identification-weight-body fat measuring module 300 identifies the user by measuring the ECG signal before measuring the biological signals. The identification-weight-body fat measuring module 300 then measures a weight of the identified user and a bioelectric impedance input from the hand electrodes 341 and the foot electrodes 342. The measured biological signals are then transmitted, e.g., wirelessly, to the biological information processing module 500.

More specifically, the identification-weight-body fat measuring module 300 performs substantially simultaneously operations of identifying a user by measuring the ECG signal, measuring a user's weight, and measuring the user's bioelectric impedance using electrodes in contact with the user's hands and feet, all while the height measuring module 200 is measuring the user's height.

The weight-body fat measuring module 340 may include the hand electrodes 341, the foot electrodes 342, the load cell 343, a weight measuring unit 344, a body fat measuring unit 345, an ADC 346, a control unit 347, and a measurement data transmitting/receiving unit 348. An identification module 320 may include an ECG measuring unit 322, and shares the hand and foot electrodes 341, 342 as ECG electrodes in order to process the ECG signal acquired from the ECG measuring unit 322 by sharing the ADC 346, the control unit 347, and the measurement data transmitting/receiving unit 348.

An operation performed in each module will now be described in greater detail.

First, the identification module 320 transmits an amplified ECG signal to the ADC 346 after amplifying by a predetermined magnitude the ECG signal input from the hand electrodes 341 and the foot electrodes 342 through the ECG measuring unit 322. An ECG record method used for measuring the ECG signal may be performed through electrodes of a right hand and a left hand by a first standard limb lead, may be performed through electrodes of a right hand and a left foot by a second standard limb lead, and may be performed through electrodes of a left hand and a left foot by a third standard limb lead.

After receiving the digitized ECG signals from the ADC 346, the control unit 347 analyzes the ECG signal, and identifies the user by comparing the analyzed ECG signal with ECG templates of registered user's. When the input ECG signal is equal to one of the registered ECG signals, the control unit 347 retrieves the identified user's personal data, e.g., a user's name, birth date, sex, blood type, basic medical history, and the like, which are stored in the biological information processing module 500 through the measurement data transmitting/receiving unit 348. As a result, biological information, e.g., fat condition information and nutritive condition information, can be measured without repetitively inputting information required for managing the user's growth and development, e.g., data regarding the user's name, birth date, sex, blood type, basic medical history, etc. In this case, combined information of weight, bioelectric impedance, body fat, etc., as well as the ECG signal acquired from the user, may be also used as information for identifying a user.

Next, an operation of each functional block constituting the weight-body fat measuring module 340 will be described.

The weight measuring unit 344 receives a user's weight data sensed by the load cell 343 when a user steps onto the identification-weight-body fat measuring module 300, amplifies the received data, and transmits the amplified data to the ADC 346. At this time, data used for measuring weight is data acquired during the few seconds before the user's hands contact the hand electrodes 341 after the user steps onto the identification-weight-body fat measuring module 300. The control unit 347 receives the digitized weight data from the ADC 346, calculates an average of the received weight data, and outputs the average as the user's weight. Further, the output weight data are transmitted, e.g., wirelessly, to the biological information processing module 500 through the measurement data transmitting/receiving unit 348.

When a user contacts both foot electrodes 342 and both hand electrodes 341 of the identification-weight-body fat measuring module 300, the body fat measuring unit 345 measures the bioelectric impedance of the user. A bioelectric impedance measuring method is a method of estimating a water amount, a muscle amount, a fat amount, etc., of a human body by measuring electric resistance or impedance of the human body when the human body contacts the electrodes 341 and 342 and a weak alternating current flows through the human body. The body fat measuring unit 345 may include a current source 3451 for flowing a predetermined current between the two electrode pairs 341 and 342, a voltage meter 3452 for measuring a voltage between the two electrode pairs 341 and 342, and an amplifier 3453 for amplifying the voltage measured in the voltage meter 3452. Bioelectric impedance data measured in the body fat measuring unit 345 is amplified by the amplifier 3453 and is transmitted to the ADC 346. A control unit 347 receives digitized bioelectric impedance data from the ADC 346 and analyzes body fat components from the received bioelectric impedance data. The analyzed body fat data is transmitted, e.g., wirelessly, to the biological information processing module 500 through the measurement data transmitting/receiving unit 348.

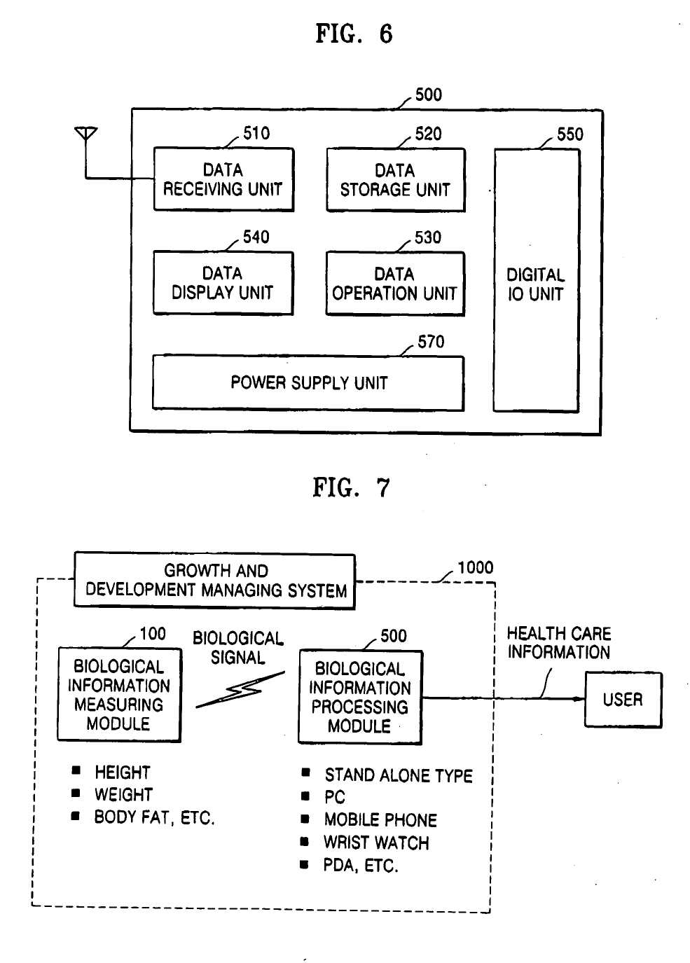

FIG. 6 is a block diagram illustrating a configuration of a biological information processing module according to an embodiment of the present invention. FIG. 7 is a block diagram illustrating an overall configuration of the system to which the biological information processing module shown in FIG. 6 is applied.

Referring to FIG. 6, the biological information processing module 500 may include a data receiving unit 510, a data storage unit 520, a data operation unit 530, a data display unit 540, a digital input and output (digital 10) unit 550, and a power supply unit 570. The biological information processing module 500 analyzes, stores, and manages the biological information transmitted, e.g., wirelessly, from the biological information measuring module 100.

The data receiving unit 510, which receives the biological information from the biological information measuring module 100, may have a radio data receiver for receiving the biological information wirelessly from the biological information measuring module 100. The data storage unit 520 sorts and stores the biological information received from the data receiving unit 510 for each user. The data operation unit 530 analyzes growth and development data of a user, e.g., a child, through a statistical process and a predetermined analyzing algorithm of the received biological information. The analyzed results by the data operation unit 530 are transmitted to the data storage unit 520 and are stored for each user, and the analyzed results or the biological information stored in the data storage unit 520 are supplied to the user by the data display unit 540. The data display unit 540 may have a speaker (not shown) for outputting relevant data audibly and a display device, e.g., liquid crystal display (LCD), or the like, for outputting the analyzed biological information visually. The digital 10 unit 550 controls input/output of the data receiving unit 510, the data storage unit 520, the data operation unit 530, and the data display unit 540. The power supply unit 570 supplies electric power required by the biological information processing module 500.

Referring to FIG. 7, the biological information processing module 500 together with the biological information measuring module 100 is included in the system 1000. The biological information processing module 500 receives, e.g., wirelessly, analyzes, and manages for each user the biological information of height, weight, body fat, etc. measured by the biological information measuring module 100. The biological information processing module 500 may be embodied in an exclusive platform, i.e., a stand alone type, for managing the user's growth and development information, or may alternatively be embodied in a mobile phone, a PDA, or a wrist-watch typed potable terminal, etc.

As a configuration and function of the growth and development managing system 1000 is extended so that the biological information processing module 500 can remotely interact with a data center (see 700 of FIG. 9) as well as the biological information measuring module 100, the system can supply fatness and nutritive condition managing services and health information managing services for any individual.

The configuration of the biological information processing module having such extended functions will now be described.

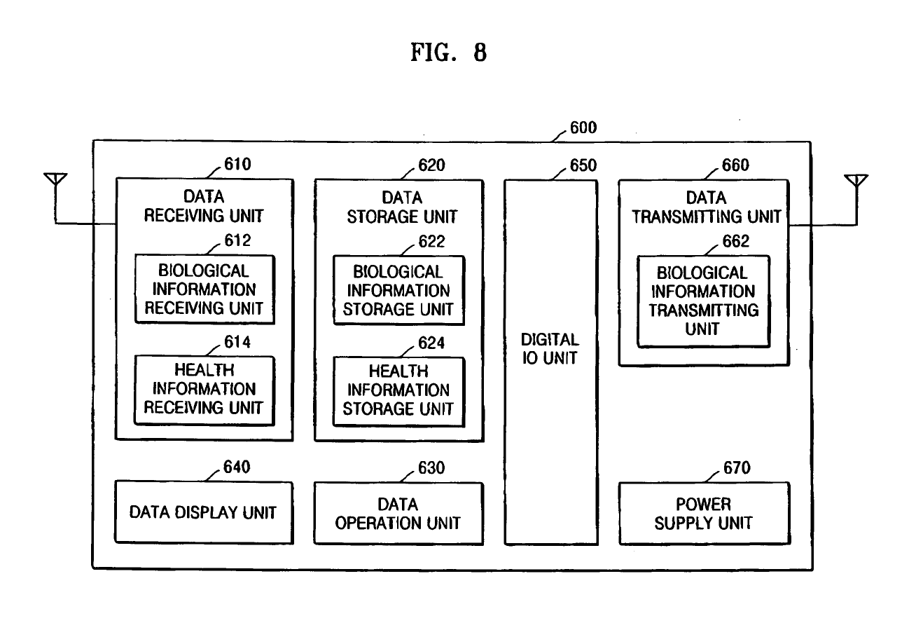

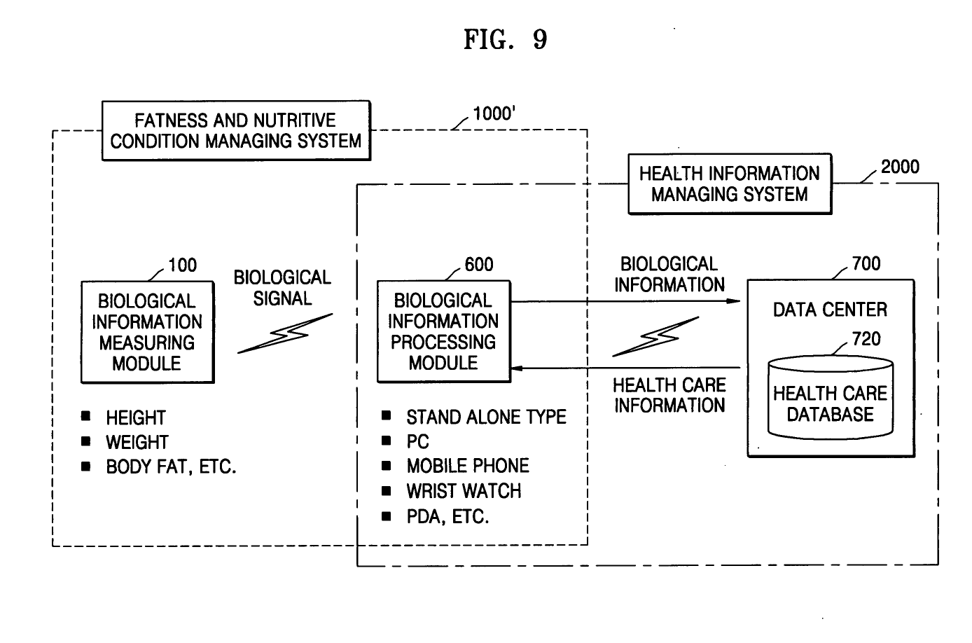

FIG. 8 is a block diagram illustrating a configuration of the biological information processing module according to another embodiment of the present invention. FIG. 9 is a block diagram illustrating a configuration of a fatness and nutritive condition managing system and a health information managing system to which the biological information processing module shown in FIG. 8 is applied.

Referring to FIG. 8, the biological information processing module 600 performs functions of analyzing the biological information transmitted, e.g., wirelessly, from the biological information measuring module 100 to manage fatness and nutritive conditions, and of storing and managing health care information for each user transmitted, e.g., wirelessly, from the data center (see 700 of FIG. 9). The biological information processing module 600 may include a data receiving unit 610, a data storage unit 620, a data operation unit 630, a data display unit 640, a digital 10 unit 650, a data transmitting unit 660, and a power supply unit 670.

The data receiving unit 610 may include a biological information receiving unit 612 and a health information receiving unit 614. The data receiving unit 610 receives the biological information transmitted from the biological information measuring module 100 and the health care information for each user transmitted from the data center 700 of FIG. 9. The data storage unit 620 may include a biological information storage unit 622 and a health information storage unit 624. The data storage unit 620 stores the biological information and the health care information for each user received from the biological information receiving unit 612 and the health information receiving unit 614. Particularly, since a user identification function is provided to the fatness and nutritive condition managing system 1000′ to which the biological information processing module 600 is applied and a health information managing system 2000, the data storage unit 620 manages the biological information and the health care information for each user according to a user recognition result.

The data operation unit 630 analyzes a fatness and nutritive condition management information of a user, e.g., a child, through a statistical process and a predetermined analysis algorithm on the received biological information. The analyzed results are then transmitted to the data storage unit 620, are stored for each user, and are transmitted, e.g., wirelessly, to the data center through the digital 10 unit 650 and the data transmitting unit 660. Alternatively, the analyzed results or the biological information stored to the data storage unit 620 may be supplied directly to a user through the data display unit 640. The data display unit 640 may have a speaker (not shown) to output related information audibly and a display device, e.g., an LCD, etc., to output the analyzed biological information visually.

The data transmitting unit 660 may include a biological information transmission unit 662 and transmits the analyzed results of the biological information to the data center 700 of FIG. 9. The digital 10 unit 650 controls the input/output of the data receiving unit 610, the data storage unit 620, the data operation unit 630, the data display unit 640, and the data transmitting unit 660. The power supply unit 670 supplies electric power required by the biological information processing module 600.

As described above, the biological information processing module 600 shown in FIG. 8 performs functions of processing and managing biological information for each user as in the biological information processing module 500 shown in FIG. 6 and may perform additional functions of exchanging and managing health information, such as customized health information for each predetermined period for each user based on height, weight, body fat, etc., with the data center 700, which may be remotely located. Therefore, the module can systematically supply the growth and development data for each user, including health-relevant information, on a corresponding user.

Referring to FIG. 9, the biological information processing module 600 together with the biological information measuring module 100 is included in the fatness and nutritive condition managing system 1000′, and the biological information processing module 600 is also included in the health information managing system 2000. The biological information processing module 600 receives, analyzes, and manages for each user biological information related to height, weight, body fat, etc. measured from the biological information measuring module 100. Further, the biological information processing module 600 together with the data center 700 is included in the health information managing system 2000. The biological information processing module 600 analyzes, stores, and manages the biological information transmitted, e.g., wirelessly, from the biological information measuring module 100, and stores and manages the health care information for each user transmitted, e.g., wirelessly, from the data center 700. The biological information processing module 600 may be embodied in an exclusive platform, i.e., a stand alone type, a mobile phone, a PDA, a wrist-watch typed potable terminal, etc. for managing the user growth and development data or fat and nutritive management data and health-relevant information.

The data center 700 may be remotely situated from the biological information processing module 600. The data center 700 may include a health care data database 720, and stores the health-relevant information for each user to a database.

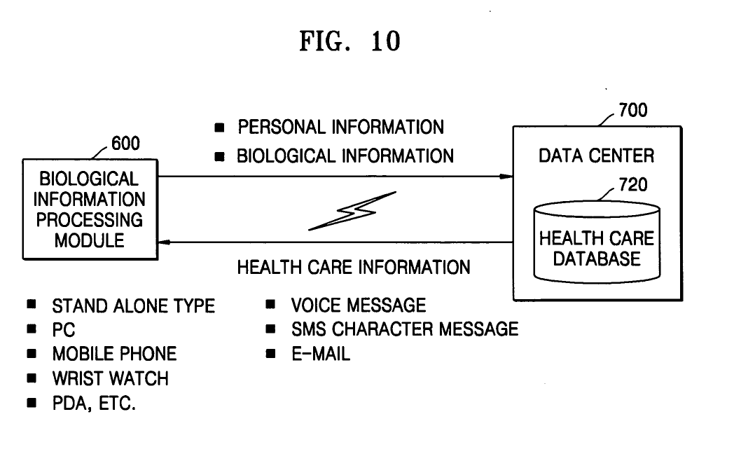

FIG. 10 is a diagram illustrating data flow between the biological information processing module 600 and the data center 700 shown in FIG. 9.

Referring to FIG. 10, the data center 700 analyzes the health care information of a user for each period, e.g., daily, weekly, monthly, based on the biological information, e.g., height, weight, body fat data, and the user personal information, e.g., sex, age, basic medical history, transmitted from the biological information processing module 600, performs statistical processes and analytic algorithms on the health care information and the user personal information, and then separates the performed results for each user and stores the separated results to the health care information database 720. Further, the data center 700 can make the biological information processing module 600 integrally supply the health care information to a user by transmitting back the health care information for each user stored in the health care information database 720 to the biological information processing module 600. The health care information may be supplied as a voice message, a short message service (SMS), an e-mail, etc., to a user or a user may retrieve relevant data by logging in the data center 700.

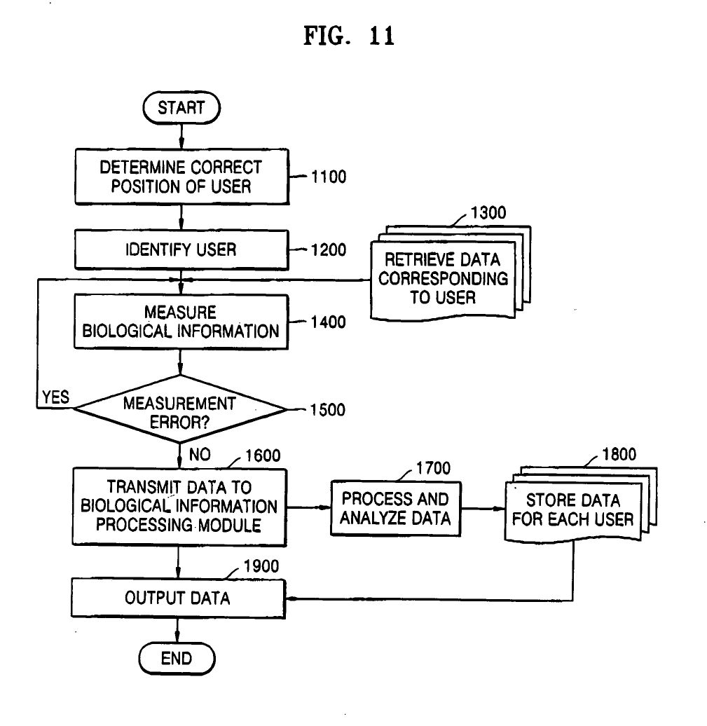

FIG. 11 is a flowchart illustrating a growth and development measuring and analyzing method to be performed in a system 1000 for managing growth and development of a child, according to an exemplary embodiment of the present invention.

Referring to FIG. 11, the system 1000 for managing growth and development of a child determines, in operation 1100, whether a user is standing in a correct position for the measurement of the biological information. In operation 1200, the system 1000 identifies a user by acquiring an ECG signal from the user through the integrated identification-weight-body fat measuring module 300. At this time, a combination of weight information, bioelectric impedance information, body fat information, etc. acquired from the user, as well as the ECG signal, may be used in the identification process. After a user is identified, in operation 1300, the registered data related to the user is retrieved, and thus a pre-process for measurement is completed.

Next, in operation 1400, biological information, e.g., height, weight, body fat data, etc., is measured by the biological information measuring module 100. In operation 1500, it is determined whether there is an error in the measured biological information. When there is an error in the measured biological information, the process returns to operation 1400 and the biological information is measured again. When there is no error in the measured biological information, in operation 1600, the measured biological information is transmitted, e.g., wirelessly, to the biological information processing module 500.

Next, in operation 1700, after the biological information processing module 500 processes and analyzes the biological information transmitted from the biological information measuring module 100, the analyzed results are stored for each user in operation 1800. The analyzed results stored in operation 1800 may be stored for each user in the biological information processing module 500, and may be stored to a database at the data center 700. In operation 1900, such biological information stored for each user is output to a user in a voice message, a SMS, an e-mail, etc., through the biological information processing module 500. Additionally, a user may retrieve relevant data by logging into the data center 700.

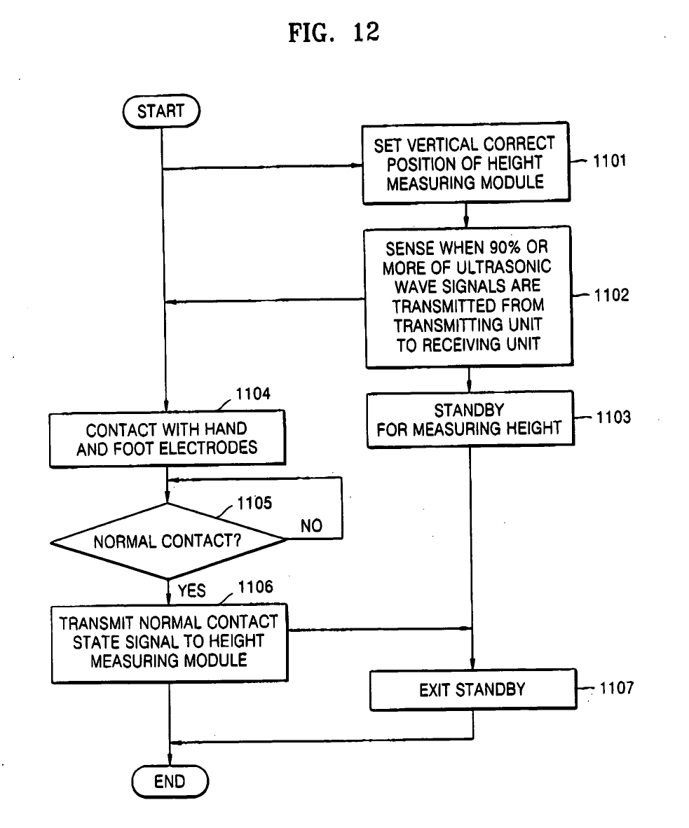

FIG. 12 is a detailed flow chart for determining whether a user is standing at a correct position shown in FIG. 11.

Referring to FIG. 12, in operation 1101, the system 1000 sets a vertical correct position of the height measuring module 200 for determining whether a user is standing at a correct position. The vertical correct position setting process performed in operation 1101 is a process of normally implementing the ultrasonic wave generating unit 212 and the ultrasonic wave receiving unit 214 on the ceiling and the floor, respectively, of a room for measuring height and is performed during initialization of the system 1000.

Next, in operation 1102, when 90% or more of ultrasonic wave signals generated from the ultrasonic wave generating unit 212 is sensed in the ultrasonic wave receiving unit 214, it is determined that the height measuring module 200 is functioning normally.

When, in operation 1102, it is determined that the height measuring module 200 is functioning normally, in operation 1103, a process of measuring height is on standby, and, in operation 1104, the user's hands and feet contact the hand and the foot electrodes 341 and 342, respectively, of the integrated identification-weight-body fat measuring module 300. Then, in operation 1105, it is determined whether the contact state of electrodes is normal by confirming whether the hand and foot electrodes 341 and 342 of the identification-weight-body fat measuring module 300 are normally contacted to a user's hands and feet. For example, if four or eight electrodes normally contact a user's hands and feet when measuring body fat, a voltage drop starts from a saturated bioelectric impedance. The contact state of the electrodes is determined to be normal if there is no abnormality a few seconds after the start of the voltage drop.

After operation 1105 and when the contact state of electrodes is determined to be normal, in operation 1106, the integrated identification-weight-body fat measuring module 300 transmits signals that the contact state of the electrodes is normal to the ultrasonic wave generating unit 212 in the height measuring module 200. In operation 1107, the height measuring module 200 in a standby mode for measurement exits the standby mode and completes preparation for measurement.

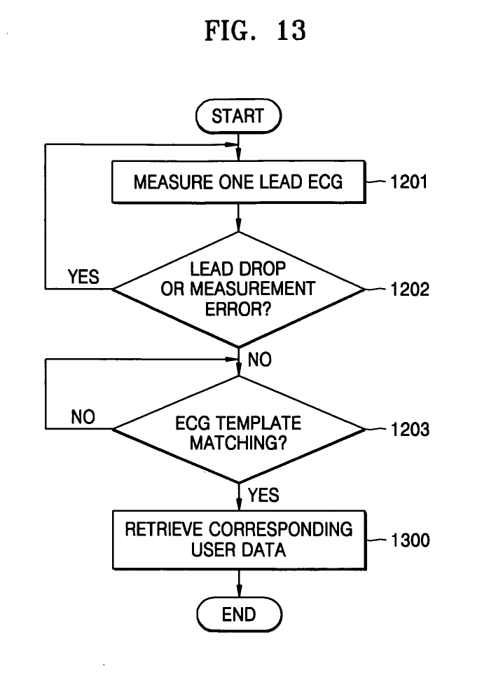

FIG. 13 is a detailed flow chart illustrating a user identifying process shown in FIG. 11.

Referring to FIG. 13, in order to identify a user, in operation 1201, the identification-weight-body fat measuring module 300 in the system 1000 measures an ECG signal through the hand electrodes 341 and the foot electrodes 342 contacted to the user's hands and feet, respectively. In operation 1202, it is determined whether leads connected to the hand and foot electrodes 341, 342 are dropped or there is an error in the measurement of the ECG signal. When there is an error in measuring the ECG signal, the process returns to operation 1201 and the ECG signal is measured again. When there is no error in the measurement of the ECG signal, it is determined in operation 1203 whether there is a correspondence with a previously stored ECG template by comparing the ECG signals measured from the ECG measuring unit 322.

In this case, the ECG templates to be used for identifying a user may be stored to a personal data storage unit in the identification-weight-body fat measuring module 300, or may be remotely stored at the biological information processing module 500 or the data center 700 depending on the system configuration. In the present invention, the personal data storage unit may interact with the biological information measuring module 100 by implementing the personal data storage unit in the biological information processing module 500. More specifically, the personal data storage unit may be included in an integrated form to the biological information processing module 500, or may be embodied in a portable electronic record media, e.g., an electronic record card.

Next, in operation 1300, when there is a corresponding ECG template, the identification-weight-body fat measuring module 300 retrieves the corresponding user's data, e.g., name, birth date, sex, blood type, basic medical history, etc. from the biological information processing module 500.

That is, in order to identify a user, the identification-weight-body fat measuring module 300 records ECG data acquired for a predetermined time by the standard limb lead of ECG, and the identification process is completed by retrieving the most interrelated user health information record through an operation to compare the recorded user ECG data with the previously input ECG templates for each user.

The ECG recording for identification continues for about twenty seconds to thirty seconds during which time a heart beat fluctuates between twenty and thirty times. When the recording is completed, the interrelationship is analyzed using a training algorithm such as a template matching and neural network, etc. QRS onset, P duration, QRS duration, R duration, S duration, ST slope, QRS p-p amplitude, T amplitude, and ST amplitude, etc. may be used as ECG matching parameters for the template matching. The ECG signal, however, is just an exemplary signal adapted for use in a user recognition method. Weight, bioelectric impedance, body fat information, and so on acquired from the user may be also used in connection with user recognition, as well as the ECG signal.

According to a user recognition method of the present invention, whenever biological information is measured, a user is automatically identified by the ECG signal, and health-relevant information as well as the growth and development information of each identified user, can be systematically managed without requiring additional input of a user's personal data.

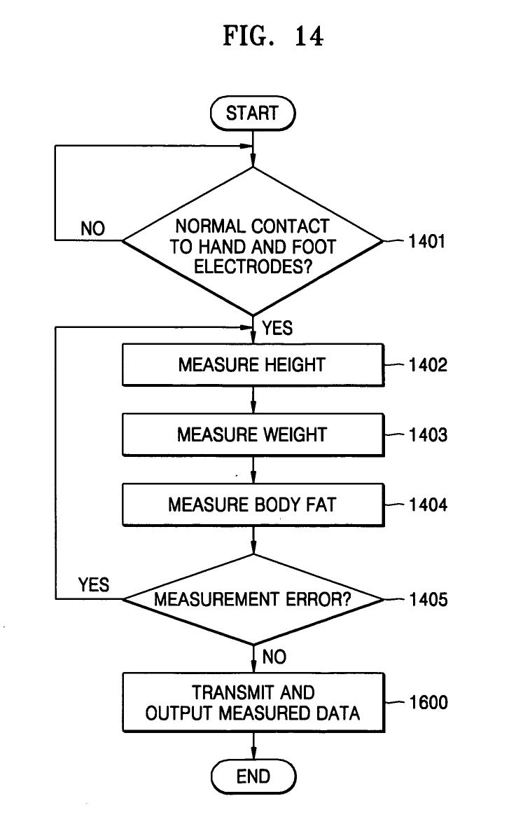

FIG. 14 is a detailed flow chart illustrating the biological information measuring process shown in FIG. 11.

Referring to FIG. 14, in order to measure the biological information, in operation 1401, the identification-weight-body fat measuring module 300 in the system 1000 determines whether a contact state of the hand electrodes 341 and the foot electrodes 342, which are contacted to a user's hands and feet, respectively, is normal. When there is an error in the contact state of the hand or foot electrodes 341 and 342, a simple voice message requesting another measurement is output. When the contact state of the hand and foot electrodes 341 and 342 is normal, in operation 1402, the user's height is measured by the height measuring module 200 mounted on the ceiling. In operation 1403, the user's weight is measured by the identification-weight-body fat measuring module 300.

In operation 1404, after a bioelectric impedance is measured from the hand and foot electrodes 341 and 342 contacted to the user's hands and feet, respectively, body fat is calculated using the measured bioelectric impedance, height information measured in operation 1402 and weight information measured in operation 1403.

Then, in operation 1405, it is determined whether there was a measurement error. When no measurement error is detected, in operation 1600, the measured data is transmitted, e.g., wirelessly, to the biological information processing module 500.

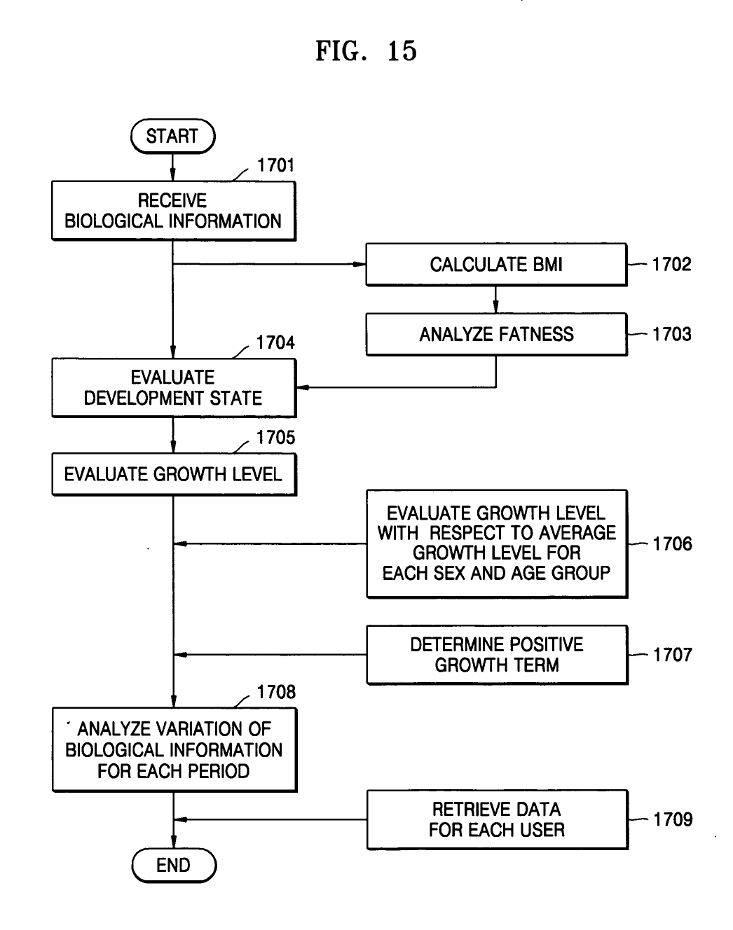

FIG. 15 is a detailed flow chart illustrating the data processing and analyzing process shown in FIG. 11.

Growth and development evaluation performed by the system 1000 is divided into a development state evaluation, a growth level evaluation, a positive growth entry term and positive growth term determination, and an analysis of variation transition in growth and development for each period as shown in Table 1.

TABLE 1

| Data processing | Method |
|---|---|
| Evaluation of development | 1) weight analysis and fatness |
| state | determination by BMI calculation (weight |
| | (kg)/ square of height (m)); weight analysis |
| | and fatness determination based on BMI |
| | table by sex and age of corresponding |
| | year |
| | 2) fatness determination by bioelectric |
| | impedance measuring method |
| Analysis of growth level | express the difference based on average |
| evaluation | height and weight by sex and age as |
| | percentage |
| | * growth level versus average height by |
| | sex and age |
| | = {(average height by sex and age-user's |
| | height)/(average height by sex and |
| | age)}*100 |
| | * growth level versus average weight by |
| | sex and age |
| | = {(average weight by sex and age-user's |
| | weight)/(average weight by sex and |
| | age)}*100 |
| Determination of positive | * the inflection point portion where the |
| growth entry term and | slope of a height increment rate curve in |
| positive growth term | the past six months, after age six years, is |
| | converted to positive (+) from negative (−) |
| | or zero (0) is determined to be a positive |
| | growth entry term, and based on this, |
| | when the slope of an increment rate curve |
| | maintains a positive slope for six months or |
| | longer or becomes a steeper positive |
| | slope, it is determined to be a positive |
| | growth term. |
| Analysis of variation in | Variation is output as a graph over a |
| biological information and | predetermined period by cumulatively |
| development state for each | summing weight, height, body fat |
| period | percentage, and fatness data for each |
| | period. |

Referring to FIG. 15, in order to evaluate the growth and development of children as shown in Table 1, in operation 1701, the biological information processing module 500 in the system 1000 receives the biological information, i.e., weight, body fat, height, etc., measured from the identification-weight-body fat measuring module 300. In operation 1702, the biological information processing module 500 calculates Body Mass Index (BMI) using Equation 1:
BMI=weight (kg)/square of height (m) (1)

After BMI is calculated, in operation 1703, weight and a degree of fatness are analyzed based on a BMI table for sex and age. The BMI table used at this time may be replaced with an updated BMI table for the current year.

Table 2 shows an exemplary BMI table.

TABLE 2

| Age | Overweight | | Obese | |
|---|---|---|---|---|
| 4 | male | 17.56 | Male | 19.29 |
| | female | 17.28 | Female | 19.15 |
| 6 | male | 17.56 | Male | 19.78 |
| | female | 17.34 | Female | 19.65 |
| 8 | male | 18.44 | Male | 21.60 |
| | female | 18.35 | Female | 21.57 |
| 10 | male | 19.84 | Male | 24.00 |
| | female | 19.86 | Female | 24.11 |
| 12 | male | 21.22 | Male | 26.02 |
| | female | 21.68 | Female | 26.67 |
| 14 | male | 22.62 | Male | 27.63 |
| | female | 23.34 | Female | 28.57 |
| 16 | male | 23.90 | Male | 28.88 |
| | female | 24.37 | Female | 29.43 |
| 18 | male | 25.00 | Male | 30.00 |
| | female | 25.00 | Female | 30.00 |

| | | |
|---|---|---|
| 18 or older | 25 to 29 | 30 > BMI |

The analysis of fatness of a child performed in operation 1703 is largely divided into determination of fatness based on the BMI table and determination of fatness based on the bioelectric impedance. More specifically, in operation 1703, after an initial determination of fatness based on the BMI table, determination of fatness based on the bioelectric impedance is selectively performed, so that more correct fatness information of a user can be obtained.

In a bioelectric impedance measuring method, a minute current, of which a user cannot sense, is flowed to the user's hands and feet after four electrodes are attached, typically on an ankle, a foot, a wrist, and a hand, to detect a voltage from the wrist and the ankle. Then, the method calculates a body fat percentage based on an electric conductivity, using the measured voltage, of a human organ.

It is known that the bioelectric impedance measuring method has a high interrelation (r=0.90-0.94) with the body fat percentage obtained through an underwater weight measuring method. However, because of a feature of a body fat percentage equation, the body fat percentage of a thin person tends to be overestimated. Therefore, in order to solve such a problem, the present invention determines fatness once again based on the bioelectric impedance of a user having been determined to be overweight or obese by the BMI, thereby enhancing reliability of health information on the user development state.

Next, after the fatness is analyzed, in operation 1704, the user development state is evaluated based on the analyzed result. In operation 1704, subjects are divided in three groups having low, normal, and high body fat percentage, to determine the development state of users belonging to each group.

Table 3 shows definitions of three groups based on the body fat percentage.

TABLE 3

| Body fat percentage | Group to be evaluated |
|---|---|
| less than 10% | Low |
| 10% to 14% | Normal |
| 15% or more | High |

Next, in operation 1705, evaluation of a user's growth level is performed. In operation 1706, the user's growth level versus average growth by sex and age is evaluated. The evaluation of the growth level performed in operation 1706 is divided into evaluation of the growth level versus average height by sex and age and evaluation of the growth level versus average weight by sex and age, and both evaluations are calculated by the following equations, respectively.
Growth level vs. average height by sex and age={(average height by sex and age−user's height)/(average height by sex and age)}*100(%) (2)
Growth level vs. average weight by sex and age={(average weight by sex and age−user's weight)/(average height by sex and age)}*100(%) (3)

As described in Equations 2 and 3, in operation 1706, average growth information (height, and weight) and a user's growth information are compared and the compared result is represented by a percentage, so that the growth level is evaluated.

Next, in operation 1707, it is determined whether the growth level corresponds to a positive growth term.

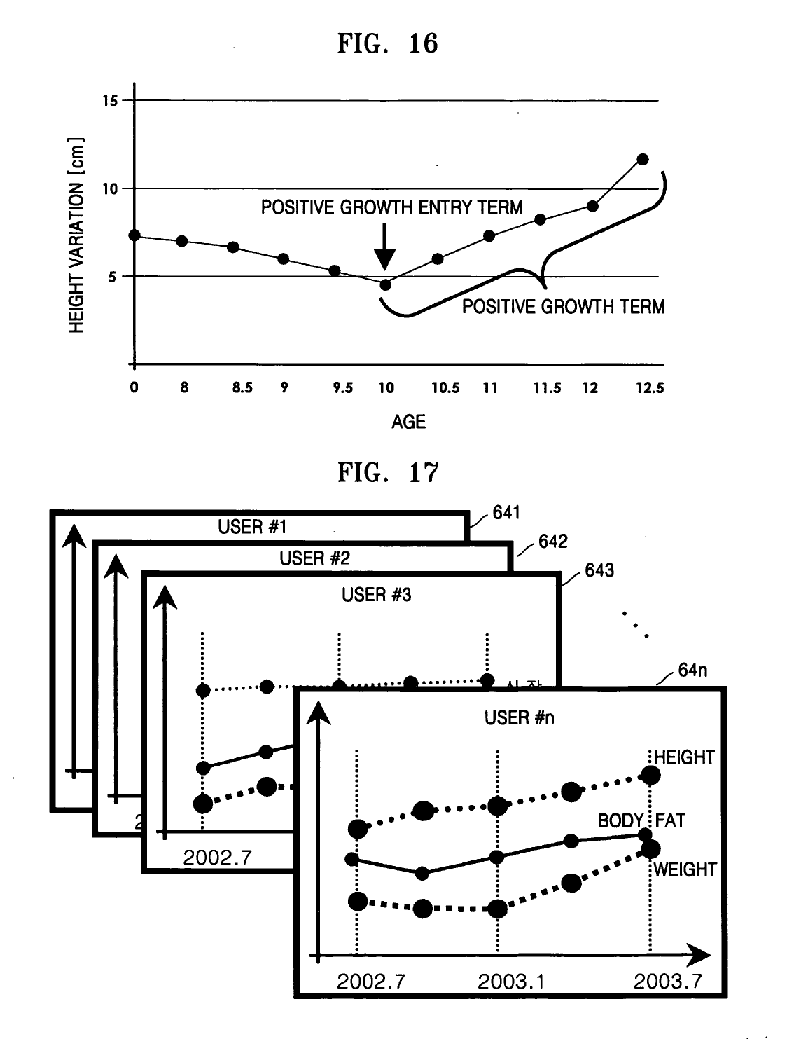

FIG. 16 is a graph illustrating the positive growth term determining process shown in FIG. 15.

Referring to FIG. 16, when a slope of a height increment rate curve for the most recent 6 month period after a user's age reaches six years is converted to a positive (+) value from a negative (−) value or zero (0), the inflection point portion is determined to be a positive growth entry term, and based on this, when the slope of the curve maintains the positive slope for six months or longer or changes to be more steeply positive, it is determined to be a positive growth term.

Referring back to FIG. 15, after a positive growth term is determined, in operation 1708, a variation in biological information and development state for each period is analyzed. In operation 1709, data for the corresponding user are retrieved.

That is, in analyzing biological information for a user, the system 1000 does not only support analysis of instant data, but also stores the analyzed results on the development state and growth level for the user. The system 1000 then cumulatively sums the analyzed results for each period and each user, and analyzes the variations of the accumulated results.

FIG. 17 is a diagram illustrating growth and development analysis displays according to an exemplary embodiment of the present invention.

Referring to FIG. 17, the system 1000 provides tendencies in the changes of the biological information for each period for each user and the analyzed results thereof. Further, the system provides health information related to the tendencies as well as an exercise prescription, diet information, etc., required for each user. Furthermore, the system can interact with the data center 700 to manage the health information remotely, so that it is possible to provide information on exercise at a sports center, information related to disease at specialized medical clinics, and be incorporated into a function of making a doctor appointment.

As described above, the system 1000 for managing growth and development of a child collects and analyzes biological information, e.g., height, weight, body fat, etc., for a user, and shows time sequential changes of the same kind of biological information which have been stored for a predetermined period for a user, 641, 642, 643, . . . , 64n, so that the system can comprehensively provide information on a child's growth and development state and information on a solution, e.g., exercise, rest, diet, etc., for an undesirable condition that is suitable for each situation when encountered. Such health information management can be expanded to adults as well as children. Furthermore, the system can minimize a size of device and space required for measuring biological signals, whereby portability and storage of the system are facilitated, so that the system can easily be used at home or while traveling.

The present invention can also be embodied as computer readable codes on a computer readable recording medium. The computer readable recording medium may be any data storage device that can store data which can be thereafter read by a computer system. Examples of the computer readable recording medium may include read-only memory (ROM), random-access memory (RAM), CD-ROMs, magnetic tapes, floppy disks, optical data storage devices, and carrier waves such as data transmission through the Internet. The computer readable recording medium may also be distributed over network coupled computer systems so that the computer readable code is stored and executed in a distributed fashion.

Exemplary embodiments of the present invention have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

1. A system for managing growth and development of a user, the system comprising:
a biological information measuring module for acquiring at least two biological signals to be used for analyzing growth and development of the user, and for identifying the user by analyzing at least one biological signal of the at least two acquired biological signals; and
a biological information processing module for evaluating a development state and a growth level of the user according to the biological signals, for storing and managing personal data and the results of the evaluation of the user.

2. The system as claimed in claim 1, further comprising a data center for receiving biological information, the personal data, and the result of the evaluation of the development state and the growth level of the user from the biological information processing module, for analyzing information required for a health condition and health care of the user, and for supplying the health-relevant information of the user at predetermined periods.

3. The system as claimed in claim 1, wherein the biological information measuring module comprises:
a height measuring module for measuring a height of the user using ultrasonic waves; and
an integrated identification-weight-body fat module for measuring an electrocardiogram, a weight, and a bioelectric impedance of the user, for identifying the user by analyzing the measured electrocardiogram, and for analyzing a body fat value from the bioelectric impedance.

4. The system as claimed in claim 3, wherein the height measuring module comprises:
a height data acquiring unit for applying the ultrasonic waves to a head portion of the user and for sensing ultrasonic waves reflected from the head portion of the user;
an analog-to-digital converter for converting the sensed ultrasonic waves into digital data;
a control unit for calculating the height of the user based on the digitized ultrasonic wave data; and
a data transmitting unit for transmitting the calculated height data to the biological information processing module.

5. The system as claimed in claim 3, wherein the identification-weight-body fat measuring module comprises:
an identification module for identifying the user by comparing the measured electrocardiogram with previously-registered electrocardiogram templates; and
a weight-body fat measuring module for measuring the weight and the bioelectric impedance of the user and for analyzing the body fat value from the measured bioelectric impedance.

6. The system as claimed in claim 5, wherein the weight-body fat measuring module comprises:
hand electrodes for contacting hands of the user and for acquiring the electrocardiogram and bioelectric impedance signal;
foot electrodes for contacting feet of the user and for acquiring the electrocardiogram and bioelectric impedance signal;
a load cell for contacting the feet of the user and acquiring a weight signal;
a weight measuring unit for amplifying the weight signal acquired by the load cell;
a body fat measuring unit for measuring the bioelectric impedance signal by flowing a predetermined amount of current through the hand electrodes and the foot electrodes and measuring voltages across the electrodes, and for amplifying the measured bioelectric impedance signal;
an analog-to-digital converter for converting the amplified weight signal and the amplified bioelectric impedance signal into digital weight data and digital bioelectric impedance data, respectively;
a control unit for calculating the weight of the user by averaging the digital weight data, and for calculating the body fat by analyzing the digital bioelectric impedance data; and
a data transmitting/receiving unit for transmitting the weight and the body fat data calculated by the control unit to the biological information processing module and for receiving the personal data of the user from the biological information processing module.

7. The system as claimed in claim 6, wherein the identification module comprises an electrocardiogram measuring unit for amplifying the electrocardiogram acquired through the hand electrodes and the foot electrodes,
wherein the electrocardiogram amplified by the electrocardiogram measuring unit are compared with the electrocardiogram templates in the control unit in order to identify the user, and the personal data is received by the data transmitting/receiving unit.

8. The system as claimed in claim 1, wherein the biological information processing module comprises:
a data receiving unit for receiving wirelessly the biological information transmitted from the biological information measuring module;
a data storage unit for storing the received biological information for the user;
a data operation unit for analyzing the biological information, for evaluating the development state and the growth level, and for analyzing tendencies of variations in growth and development of the user by period;
a data display unit for outputting the analyzed result to the user;
a digital input and output controller for controlling the data input and output of the data receiving unit, the data storing unit, the data operation unit, and the data display unit; and
a power supply unit for supplying electric power to the biological information processing module.

9. The system as claimed in claim 2, wherein the biological information processing module further comprises;
a data transmitting unit for transmitting the biological information, the personal data, and the evaluated result of the development state and the growth level of the user to the data center; and
a data receiving unit for receiving the health-relevant data supplied from the data center and for providing the health-relevant data to the user.

10. The system as claimed in claim 1, wherein the biological information processing module is provided in one selected from the group consisting of a stand-alone type device, a personal computer, a mobile phone, a wrist watch, and a personal digital assistant.

11. The system as claimed in claim 2, wherein the data center comprises a health care database for storing the health care data for the user, and the health care data is supplied to the biological information processing module by one selected from the group consisting of a voice message, a short message service, and an electronic mail.

12. A method of managing growth and development of a user, the method comprising:
determining whether the user is positioned at a correct position for measurement of biological information;
identifying the user based on at least one biological signal acquired from an integrated identification-weight-body fat measuring module;
retrieving previously registered data of the identified user and measuring biological information of the user;
transmitting the measured biological information to a biological information processing module; and
evaluating a development state and a growth level of the user in response to the biological information and storing the result of the evaluation of the user.

13. The method as claimed in claim 12, further comprising:
transmitting the biological information, personal data, and the result of the evaluation of the development state and growth level of the user to a data center; and
receiving and analyzing the biological information, the personal data, and the result of the evaluation of the development state and growth level of the user, and supplying the analyzed health-relevant information at a series of predetermined periods to the biological information processing module.

14. The method as claimed in claim 12, wherein evaluating the development state and a growth level of the user comprises:
calculating a body mass index based on the biological information and analyzing a weight and a fatness based on a body mass index table;
analyzing the weight and the fatness of the user based on bioelectric impedance measured from the user when the fatness is above a predetermined value;
evaluating the growth level of the user based on average heights and average weights for each sex and age;
determining a positive growth entry term and a positive growth term in consideration of an increment rate in height of the user; and
analyzing variation of the biological information by period by analyzing a tendency of the analyzed results for a predetermined period.

15. The method as claimed in claim 14, wherein after an age of the user is over six years old, the positive growth entry term is represented by an inflection point where a slope of a height increment rate curve for a period of the latest six months is converted into a positive (+) value from a negative (−) value or zero (0).

16. The method as claimed in claim 14, wherein the positive growth term is a term when the slope of the curve maintains the positive slope for six months or longer or when the slope of the curve becomes steeper with respect to the positive growth entry term.

17. A computer readable recording medium on which a program for executing a method according to claim 12 is recorded.

18. A computer readable recording medium on which a program for executing a method according to claim 13 is recorded.

19. A computer readable recording medium on which a program for executing a method according to claim 14 is recorded.

20. A computer readable recording medium on which a program for executing a method according to claim 15 is recorded.

21. A computer readable recording medium on which a program for executing a method according to claim 16 is recorded.
